(19) United States
(12) Patent Application Publication
(10) Pub. No.: US 2008/0214464 A1
(43) Pub. Date: Sep. 4, 2008

(54) Method for the Production of Biologically Active Rhngf

(75) Inventors: Lilia Filippa Anna Alberghina, Milan (IT); Anna Maria Colangelo, Milan (IT); Enzo Martegani, Milan (IT)

Correspondence Address: NIXON & VANDERHYE, PC, 901 NORTH GLEBE ROAD, 11TH FLOOR, ARLINGTON, VA, 22203, US

(73) Assignee: BLUEPRINT BIOTECH Srl, Milano (IT)

(21) Appl. No.: 12/067,899

(22) PCT Filed: Sep. 23, 2005
(86) PCT No.: PCT/IB2005/053159

§ 371 Date: Apr. 4, 2008

(51) Int. Cl. A61K 38/18; C07K 14/48; C12P 21/00; C12N 5/10

(52) U.S. Cl. 514/12; 530/399; 435/69.4; 435/325

(57) ABSTRACT

The present invention relates to a new rhNGF (recombinant human Nerve Growth Factor) where said rhNGF is characterized by the fact that it presents an in vitro and in vivo activity comparable to that of the native murine NGF. The present invention also relates to the process for the production of said rhNGF, said process adapted for production on middle and large scale, and the modified cells capable of producing said rhNGF.

The present invention relates to a new rhNGF (recombinant human Nerve Growth Factor) where said rhNGF is characterized by the fact that it presents an in vitro and in vivo activity comparable to that of the native murine NGF. The present invention also relates to the process for the production of said rhNGF, said process adapted for production on middle and large scale, and the modified cells capable of producing said rhNGF.

### BACKGROUND

NGF (Nerve Growth Factor) is the founding member of the neurotrophin family and was initially identified in extracts of mice sarcomas for its capability to stimulate the survival and differentiation of sympathetic and sensory neurons of the peripheral nervous system (Levi-Montalcini R, Ann. N. Y. Acad. Sci. 55: 330-343, 1952).

Since its discovery, the richest source of NGF are the submaxillary glands of male mice, although in the vertebrates other sources of NGF include the peripheral tissues innervated by NGF-dependent neurons. For what concerns humans, there is a patent (U.S. Pat. No. 5,210,185) related to a process for the purification of beta hNGF from 7S hNGF extracted from placenta. However, in the said process chemicals were used that interferes with the activity of the extracted NGF, and no examples or data that provide information regarding the in vivo activity of the beta NGF obtained are provided in the said patent. Indeed, although the patent was filed in 1989, up to date no human NGF is commercially available besides the recombinant protein.

Several studies have then demonstrated that this neurotrophic factor is also expressed in the central nervous system, where it plays a key role for the development of the cholinergic neurons, whose degeneration is one of the main neuropathological features of Alzheimer's disease and responsible for the cognitive deficits associated with this disorder.

The experimental evidences obtained up to date in vitro and in vivo indicate that beta NGF possesses an enormous therapeutic potential for both Alzheimer and several forms of peripheral neuropathies, as in part supported also by the results of some phase II clinical trials.

Thereafter, murine NGF has been used for a large number of studies which have greatly improved the knowledge of the biological activity and the functional role of NGF. Indeed, murine NGF was found to be effective in the treatment of human cutaneous and corneal ulcers, as well in some forms of vasculite. Moreover, NGF seems to be also involved in the development and regulation of the immune system and, in fact, a certain effectiveness was demonstrated also in animal models of experimental allergic encephalomyelitis.

Up to date, most of the experimental evidences have been obtained by using NGF extracted from submaxillary glands of male mice.

Murine NGF extracted from mice submaxillary glands is a multiprotein complex of about 130 kDa which is formed by three different subunits (α, β e γ) associated in a pentameric complex α2βγ2 (7S NGF). Only the β subunit (2.5S NGF) is biologically active and is the molecule responsible for the effectiveness reported in the aforementioned clinical applications. According to some studies, NGF is not present in all vertebrates as a multiprotein complex composed by alpha beta and gamma subunits. About humans, according to the aforementioned US patent, NGF seems to be present in its native form as 7S NGF, however, no further studies have reported the extraction of NGF from human tissues and, therefore, it is not clear whether in humans hNGF in its native form is present as a 7S complex or in other conformations. What is known is that, also in humans, the beta subunit is the biologically active molecule as neurotrophic factor.

However, the murine 2.5S NGF is certainly not the pharmacological molecule suitable for clinical applications on large scale and, as mentioned above, the method described in U.S. Pat. No. 5,210,185 did not bring, in 16 years, to any commercial production of NGF or NGF-based medicaments. Hence, the pharmacological interest developed about this protein renders the production of a recombinant human NGF (rhNGF) that might be properly used for therapeutic applications of said molecule necessary.

The recombinant DNA technologies, allow for the production of a large number of recombinant human proteins for therapeutic purposes, however, up to date all the attempts to produce recombinant human NGF microorganisms such as S. cerevisiae (Kanaja et al, Gene 83:65-74, 1989) and E. coli (Negro et al., Gene 110:251-256, 1992) did not give the expected results.

Human β-NGF is a 26 kDa homodimer formed by two β chains of 120 aa that are cleaved from a 241 aa precursor molecule including a pro-sequence of 103 aa and a signal sequence of 18 aa required for transport and post-traductional processing in the endoplasmatic reticulum. Moreover, a not negligible data is that the biological activity of β-NGF appears to be dependent upon the correct formation of three intramolecular disulphide bridges and a cystein-knot.

Given the biological activity of the beta subunit, the attempts to produce human NGF focused on said subunit, therefore, in the art when referring to rhNGF one usually means the beta subunit of said molecule.

In spite of the large number of examples for production of heterologous proteins in prokaryotes, the lack of success for production of rhNGF in E. coli seems to depend on the incapability of the prokaryotic systems developed to correctly process the NGF precursor by making the correct proteolitic cleavages. Moreover, in these systems also the expression of the DNA sequence encoding for the mature protein determines the synthesis of a biologically inactive protein forming inclusion bodies.

The lack of biological activity seems to depend from the lack of formation of the correct disulphide bridges and from the subsequent formation of a protein structure different from that of the native human protein. However, also the solubilization of the inclusion bodies produced in these systems and the refolding of the protein into a biologically active tertiary structure determines low production yields and poor biological activity, thus leaving open the need of producing a rhNGF that might compete with the murine NGF both in terms of effectiveness and in terms of production yields.

Production of rhNGF has also been obtained in insect (Barnett et al., J. Neurochem. 57: 1052-1061, 1991; U.S. Pat. No. 5,272,063) and mammalian cells (Iwane et al., Biochem. Biophys. Res. Commun. 171: 116-122, 1990; U.S. Pat. No. 5,639,664; Gray and Ullrich, Genentech Inc., U.S. Pat. No. 5,288,622) with better results in terms of production yields of biologically active recombinant protein. Nevertheless, up to date most of the experimental evidence regarding the biological activity of the rhNGF arises from in vitro studies. The in vivo data available up to date, did demonstrate that in clinical trials of peripheral neuropathies the recombinant protein did not possess an activity comparable to that of the mouse NGF. This lead the pharmaceutical companies involved in the clinical trials with the rhNGF produced as described in the aforementioned reports, to discontinue the specific project regarding the rhNGF (Apfel S C, Int. Rev. Neurobiol. 50: 393-413, 2002).

As shown in the following table 1, the in vivo activity of the commercial rhNGF is not comparable to that of 2.5S mNGF, equal moles of both 2,5S NGF and beta rhNGF being used. In fact, the 2.5S murine NGF presents, in parallel experiments, an activity higher than the activity given by the commercially available homodimer beta rhNGF.

In the table reported hereinbelow, “control NI” stands for untreated mice, which did not suffer the “trauma” of the injection, while “control” stands for mice that were injected, as the treated mice, but with a molecule having the same molecular weight as the beta rhNGF but devoid of activity.

TABLE 1

| | | Body | Body | | | |
|---|---|---|---|---|---|---|
| | | weight | weight | | | |
| | | at birth | 9 days | Incisives | | In vitro |
| Molecule | Neurons/GCS | (gr) | (gr) | eruption | Eyelid opening | effects |
| Control | 25.300 ± 350 | 5.3 | 27.4 | 11 | 14 | − |
| NI | | | | | | |
| Control | 25.000 ± 367 | 5.4 | 27.5 | 11 | 14 | − |
| 2.5 S | 40.260 ± 500 | 5.1 | 24.4 | 12 | 14 | +++ |
| mNGF | | | | | | |
| rhNGF | 30.570 ± 480 | 5.3 | 22.3 | 12 | 14 | +++ |
| Sigma | | | | | | |
| rhNGF | 28640 ± 650 | 5.3 | 22.0 | 11 | 14 | ++(+−) |
| Alomone | | | | | | |

In vivo tests in neonatal mice (n=5 each experimental group) consisted in a daily administration of 5 μg/g body weight in 50 μl of physiologic solution for 5 consecutive days. Some animals were sacrificed the day after the last injection in order to count the number of cells and evaluate neuronal survival; the rest of the animals were sacrificed at post-natal day 9 to evaluate the effect of the molecule on weight development, incisive eruption and eyelid opening.

In vitro tests were carried out on sensory ganglia from chicken embryos; +++ indicates a neurite outgrowth after 24 hr in culture having the same length as the diameter of the ganglion; ++(±) indicates neurite outgrowth after 24 hr in culture equal to 75% or less of the diameter of the ganglion.

Given the poor activity in vivo of the rhNGFs produced up to date and the presence of undesirable side effects such as local hyperalgesia reported by the patients during the clinical trials, at present there is no commercial rhNGF that might be used as a medicament, notwithstanding the presence on the market of some rhNGFs that can be used exclusively for research purposes.

Therefore, until today a rhNGF that might efficiently replace the murine NGF and that might be used as a commercial medicament has not yet been produced, although NGF and its pharmacological potential have been known for many decades.

All these evidences do hence point out the need of developing systems capable of producing a beta rhNGF having a biological activity comparable to the one of the 2.5S murine NGF not only in vitro, but also in vivo. Moreover, it would be desirable that such a system of production of said biologically active rhNGF might be reproducible on middle-large scale, in order to allow the obtainment of amounts of protein having the aforementioned properties, sufficient non only for further clinical trials, but also for its therapeutic applications.

### SUMMARY OF THE INVENTION

In the present invention a system for the production of beta rhNGF was developed, in which the protein is produced in mammalian cells and the said protein, not only is directly released in the culture medium (therefore extraction procedures from cells, which can consequently cause the contamination of the product with undesired cellular material, are not required), but also it presents, unlike other known rhNGF, biological activities, both in vitro and in vivo, comparable, between 90 and 100%, to those given by the 2.5S subunit of the native murine NGF. In addition, although the systems for protein production in mammalian cells are generally characterized by low yields, the procedure of the invention allows to obtain high production yields and it can be further improved to increase by 100 fold the yield obtained with the basic procedure described below.

Objects of the invention are, therefore, a beta rhNGF having biological activities, both in vitro and in vivo, higher than 76% of those given by the 2.5S subunit of the native murine NGF at least in the tests reported below, said beta rhNGF as medicament and the pharmaceutical compositions comprising said rhNGF, the process described below for the production of said beta rhNGF, the rhNGF obtainable form said process, said process further improved for middle or large scale production of said protein and mammalian cells transformed with said method being capable to release in the culture medium particularly high amounts of said beta rhNGF.

Given the activity of the 2.5S murine NGF as 100% in the different tests of activity, the beta rhNGF object of the invention presents biological activities higher than 76% of those given by the 2.5S subunit of the native murine NGF in the following assays:

a. evaluation of PC12 pheochromocytoma cells differentiation into sympathetic-like neurons induced by incubation with the beta rhNGF of the invention, as compared to incubation with equal amounts of 2.5S mNGF;

b. evaluation of survival and differentiation of dorsal root ganglia (DRG) prepared from 7-9 days old chick embryos and/or sympathetic paravertebral ganglia explanted from 10-12 days old chick embryos after incubation with the beta rhNGF of the invention, as compared to incubation with equal amounts of 2.5S mNGF;

c. evaluation of phosphorylation of the high affinity trkA receptor induced in PC12 cells by the beta rhNGF of the invention, as compared to equal amounts of 2.5S mNGF and;

d. evaluation of the induction of hypertrophy of superior cervical ganglia, degranulation of mast cells and regulation of Substance P levels and high affinity trkA receptor expression levels in cutaneous tissues of newborn mice treated with the beta rhNGF of the invention, as compared to equal amounts of 2.5S MNGF. The tests are carried out by comparing the activity of the beta rhNGF to that of 2.5 mNGF expressed, for example, as percentage. Calculation of the percentage of activity of the rhNGF is dependent upon the kind of test being used. For example, in the case of trkA phosphorylation assay, the activity of the rhNGF is determined by means of densitometric analysis of the bands corresponding to p-trkA and by calculating the percentage of the values obtained in the samples corresponding to the treatment with the rhNGF against the value obtained with the 2.5S MNGF, the latter posed equal to 100%. For the differentiation assay, on the other hand, the activity of the rhNGF is evaluated as percentage of the 2.5S mNGF posed as 100% and considering the length of neurite processes, the number of differentiated cells versus the total number of cells and the time required to obtain that level of differentiation.

Object of the invention is also said beta rhNGF as a medicament, object of the invention are also pharmaceutical compositions comprising pharmacologically effective doses of said beta rhNGF together with suitable pharmacologically acceptable excipients depending on the pharmaceutical composition selected.

The process, object of the invention, for the production of beta rhNGF having the aforementioned properties includes the following steps:

i) the construction of an expression vector suitable for expression in mammalian cells and comprising a cDNA sequence encoding the exone 3 of the human NGF gene, said cDNA sequence including a sequence encoding the beta chain of mature human NGF (120 aa), a sequence encoding the prosequence of the beta chain of human NGF (103 aa) and a sequence encoding the signal sequence of the beta chain of human NGF (18 aa);

ii) the transformation of mammalian cells with said vector;

iii) the selection of cellular clones capable to secrete beta rhNGF having biological activities higher than 76% of those given by the 2.5S subunit of the native murine NGF in the following assays:

a. evaluation of PC12 pheochromocytoma cells differentiation into sympathetic-like neurons induced by incubation with the beta rhNGF of the invention, as compared to incubation with equal amounts of 2.5S mNGF;

b. evaluation of survival and differentiation of dorsal root ganglia (DRG) prepared from 7-9 days old chick embryos and/or sympathetic paravertebral ganglia explanted from 10-12 days old chick embryos after incubation with the beta rhNGF of the invention, as compared to incubation with equal amounts of 2.5S mNGF;

c. evaluation of phosphorylation of the high affinity trkA receptor induced in PC12 cells by the beta rhNGF of the invention, as compared to equal amounts of 2.5S mNGF and;

d. evaluation of the induction of hypertrophy of superior cervical ganglia, degranulation of mast cells, and regulation of Substance P levels and high affinity trkA receptor expression levels in cutaneous tissues of newborn mice treated with the beta rhNGF of the invention, as compared to equal amounts of 2.5S mNGF;

iv) cultivation of the cells selected at point iii) and recovery of said beta rhNGF directly from the culture medium.

Object of the invention are also the mammalian cells transformed and selected as indicated in the previous process, representative samples of said cells being the cells obtained according to the process of the invention hNGF-HeLa-BALM1 deposit number CBA PD 05004; cells hNGF-MEF-BALM2 deposit number CBA PD 05002; and the cells hNGF-MEF-BALM3 deposit number CBA PD 05003 deposited at the Centro di Biologie Avanzate (CBA) of Genova (Italy) on the 22^{nd} of Jun. 2005.

Here are provided some of the abbreviations used for this description:

rhNGF=recombinant human Nerve Growth Factor;

MNGF=murine Nerve Growth Factor (murine 2.5S NGF or beta-NGF);

trkA=high affinity NGF receptor;

DRG=Dorsal Root Ganglia;

SCG=Superior Cervical Ganglion;

SP=Substance P.

Sympathetic-like neurons=cells showing some properties characteristic of sympathetic neurons such as neurite processes similar to those of the sympathetic neurons and some neurotransmitters like dopamine and noreprinephrine.

### DETAILED DESCRIPTION OF THE FIGURES

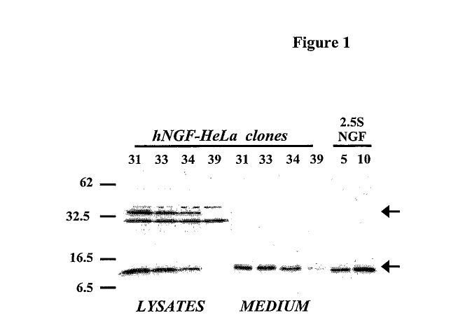

FIG. 1 shows the results of a Western Blot analysis of the beta rhNGF of the invention.

Several clones of hNGF-HeLaTetOff and hNGF-MEF TetOff cells obtained and selected according to the process of the invention were cultured in 25 cm² flasks. For Western Blot analysis of the rhNGF levels daily produced and secreted, cell lysates (5 μg) and proteins in the culture medium (5 μl) were separated by 12% SDS-PAGE and transferred onto nitrocellulose membrane. Bands of rhNGF were identified by probing the membrane with an anti-hNGF antibody (H-20, Santa Cruz Biotechnology) and quantified by densitometric analysis (Scion Image Software) by using known amounts of 2.5S MNGF (5 and 10 ng) loaded on the same gel. Arrows indicate the bands corresponding to the mature rhNGF and the pro-NGF species, the latter being present in the cell lysates only. The blot is representative of several experiments with similar results.

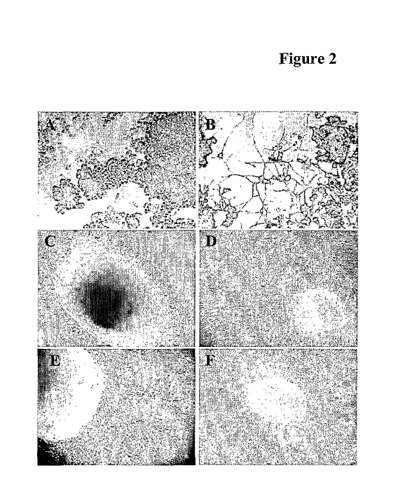

FIG. 2 demonstrates that the rhNGF of the invention induces differentiation PC12 cells and DRG. PC12 cells were exposed to the rhNGF of the invention produced by hNGF-HeLa-BALM1 cells (5 ng/ml) (panel B) and neurite outgrowth was evaluated by contrast phase microscopy after 16-18 hr. As a control, PC12 cells were incubated with an equal volume of culture medium from Mock cells (panel A).

Panels E and F show DRG explanted from 8-days old chicken embryos cultured in the presence of the rhNGF of the invention produced by hNGF-HeLa-BALM1 cells (5 ng/ml) for 48 and 96 hr, respectively. As a control, ganglia were cultured in the presence of an equal volume of culture medium from Mock cells (panel C) or 2.5S mNGF (5 ng/ml) (panel D). Treatments were repeated every 3 days and DRG differentiation and survival was monitored and recorded under a reversed microscope Olympus CX40 (20×) equipped with an Olympus camera.

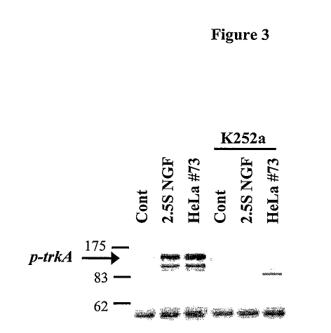

FIG. 3 shows that the rhNGF of the invention induces trkA phosphorylation in PC12 cells.

PC12 cells were incubated for 5 minutes with the rhNGF of the invention (5 ng/ml) produced by hNGF-HeLa-BALM1 cells. Phosphorylation levels were analyzed by immunoprecipition of 300 μg of total proteins with an anti-pan-trk antibody (C-14, Santa Cruz Biotechnology) and incubation with protein A. Immunocomplexes were then separated on 7.5% SDS-PAGE and transferred onto nitrocellulose membrane. Bands corresponding to p-trkA were identified by probing the membrane with the anti-p-Tyr antibody (PY99, Santa Cruz Biotechnology). As a control, PC12 cells were treated for 5 minutes with 2.5S mNGF (5 ng/ml). Where indicated, inhibition of trkA was carried out by incubating cells with K252a (100 nM, Calbiochem) for 10 minutes prior to addition of the rhNGF of the invention or 2.5S mNGF. Position of the p-trkA species is indicated by the arrow.

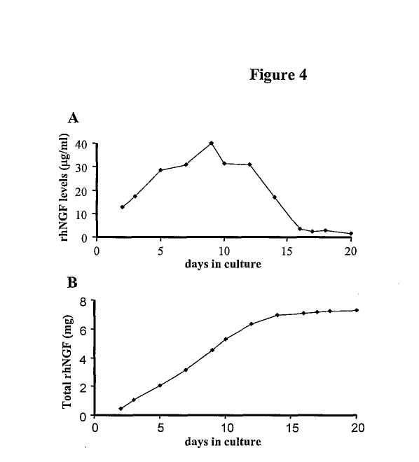

FIG. 4 shows the levels of the rhNGF of the invention produced by hNGF-HeLa-BALM1 cells in the MiniPERM.

Cells hNGF-HeLa-BALM1, deposit number CBA PD 05004 were cultured in 35 ml of DMEM supplemented with 5% FBS, and the conditioned medium harvested every 24-48 hr. The levels of rhNGF of the invention released into the medium were analyzed by Western Blot followed by densitometric analysis of the bands as described in the examples. In panel A the production profile (μg/ml) of one miniPERM is reported, while panel B shows the time-course of total production of the rhNGF of the invention (mg) in the same miniPERM system in culture for 20 days.

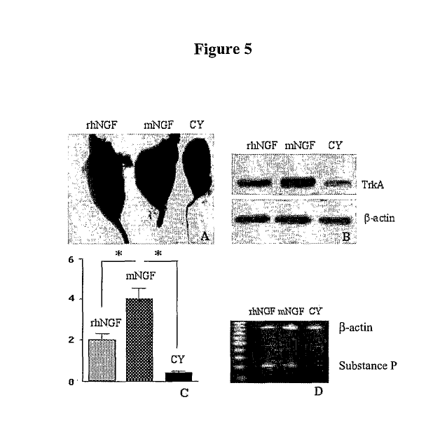

FIG. 5 shows the effects of the rhNGF of the invention in newborn mice.

Panel A: Hypertrophy of SCG from mice treated for 5 days with the rhNGF of the invention (5 μg/g body weight) or 2.5S MNGF versus control mice (CY) Ganglia were observed after staining with toluidine Blu (magnification 15×).

Panel B: Example of Western Blot analysis of trkA levels in cutaneous tissues following injection of rhNGF of the invention, 2.5S mNGF or CY. Total proteins (30 μg) were separated by 12.5% SDS-PAGE, transferred onto PVDP membrane and probed with an anti-trkA antibody (763, Santa Cruz Biotechnology). The beta-actin band was used as internal control.

Panel C: RT-PCR ELISA of SP mRNA content in the skin of mice treated with the rhNGF of the invention or 2.5S mNGF. Data are expressed as mean values ±SD. *, P<0.05 vs. control (CY).

Panel D: Example of gel stained with ethidium bromide showing the SP mRNA levels normalized by the beta-actin band.

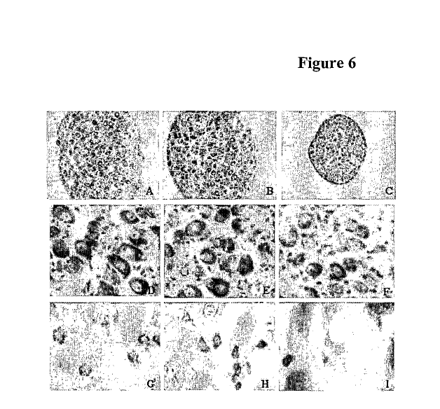

FIG. 6 shows the histological analysis of SCG and cutaneous tissues.

Mice were treated with the rhNGF of the invention or 2.5S mNGF and ganglia were dissected, fixed and stained with toluidine blu. Panels A-C show the hypertrophy of SCG induced by the rhNGF of the invention (panel A) and mNGF (panel B), as compared to ganglia from mice treated with CY (panel C) at low magnifications (180×).

Panels D-F show sections of SCG (higher magnification, 450×) from mice treated with the rhNGF of the invention (panel D), mNGF (panel E), versus those from mice treated with CY (panel F).

Panels G-H show histological sections of cutaneous tissues stained with toluidine blue showing mast cells distribution and degranulation in proximity of the injection sites of mice treated with the rhNGF of the invention (panel G), 2.5S mNGF (panel H) and CY (panel I).

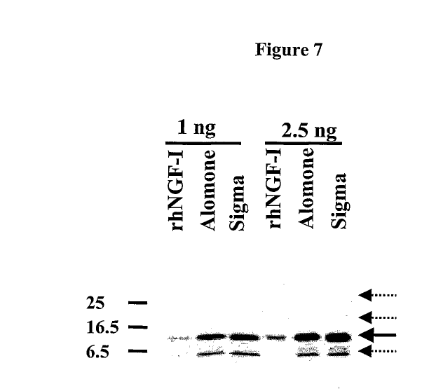

FIG. 7 shows the results of a Western Blot analysis of the beta rhNGF of the invention and rhNGF from Alomone Lab. and Sigma.

Equal amount of the different rhNGF (1 and 2.5 ng) were separated by 12% SDS-PAGE and transferred onto nitrocellulose membrane. Bands of rhNGF were identified by probing the membrane with the anti-hNGF antibody H-20 (Santa Cruz Biotechnology). The amounts of rhNGF from Alomone Lab. and Sigma were calculated by appropriate dilutions according to the amounts indicated on the vial, while those of the rhNGF of the invention were determined by Western Blot analysis performed in the same conditions and calculated on a standard curve with 2.5S MNGF (Promega) loaded on the same gel. The filled arrow indicates the band corresponding to mature rhNGF, whereas broken arrows indicate other lower and higher molecular weight bands that are absent in the rhNGF of the invention. Higher molecular weight bands are present in the rhNGF from Sigma and are more evident in the Western Blots performed with higher amounts of protein. The blot is representative of several experiments with similar results.

### DETAILED DESCRIPTION OF THE INVENTION

As already indicated, the beta rhNGF of the invention presents biological activities comparable to those given by equal doses of 2.5S mNGF at least in the following assays:

a. evaluation of PC12 pheochromocytoma cells differentiation into sympathetic-like neurons induced by incubation with the beta rhNGF of the invention, as compared to incubation with equal amounts of 2.5S MNGF;

b. evaluation of survival and differentiation of dorsal root ganglia (DRG) prepared from 7-9 days old chick embryos and/or sympathetic paravertebral ganglia explanted from 10-12 days old chick embryos after incubation with the beta rhNGF of the invention, as compared to incubation with equal amounts of 2.5S mNGF;

c. evaluation of phosphorylation of the high affinity trkA receptor induced in PC12 cells by the beta rhNGF of the invention, as compared to equal amounts of 2.5S mNGF and;

d. evaluation of the induction of hypertrophy of superior cervical ganglia, degranulation of mast cells and regulation of Substance P levels and high affinity trkA receptor expression levels in cutaneous tissues of newborn mice treated with the beta rhNGF of the invention, as compared to equal amounts of 2.5S mNGF.

Said biological activities shall be higher that 76% of those given by equal doses of 2.5 MNGF in all the aforementioned tests. In particular, in test d., representing the in vivo activity of the molecule of the invention, said activity will be advantageously comprised between 80 and 100% of the 2.5 MNGF used as reference.

In one particularly advantageous embodiment, the activity will be comprised between the 90 and 100% in all the tests described above.

For the purposes of the invention, said tests can be carried out according to all the processes known to the person skilled in the art provided that they are always performed by using equal amounts and/or concentrations (for example expressed as Molarity) of the beta rhNGF of the invention and 2.5S mNGF. In fact, given the fact that the neurotrophic activity is localized in the 2,5S MNGF subunit and in the beta rhNGF subunit, the comparison of biological activity shall be done by using equal amounts of beta rhNGF and 2.5S MNGF so that the ratio between biologically active subunits will be equal.

The biological activity in vitro can be analyzed by using PC12 pheochromocytoma cells (Greene L. A. & Tischler A. S., Proc. Natl. Acad. Sci. USA 73: 2424-2428, 1976). Said cells represent the in vitro neuronal system generally used to analyze the NGF signal transduction as well the biochemical and morphological responses induced by this neurotrophic factor.

Another neuronal system is represented by both dorsal root ganglia (DRG) prepared from 7, 8 or 9 days old chick embryos and paravertebral sympathetic ganglia prepared from 10, 11 or 12 days old chick embryos.

In one embodiment of the invention the test at point a. may be carried out by evaluating the differentiation of the aforementioned PC12 cells into sympathetic-like neurons with formation of long neurite processes. Differentiation can be expressed, for example, as percentage of the number of cells with neurite processes within a certain interval of time, or in terms of effectiveness on differentiation always within a certain time, as compared to a control system. In the present invention, the activity of the 2.5S mNGF in the same experimental conditions was used as positive control, i.e. maximum NGF activity equal to 100%.

The differentiation response can be induced after incubation of cells with concentrations of beta rhNGF, or 2.5S mNGF, comprised between 1 and 100 ng/ml, in particular, between 5 and 20 ng/ml, and can be observed after an incubation time between 8 and 48 hours, in particular for a time comprised between 16 and 24 hours.

In a further embodiment of the invention, the test at point b. may be carried out on explants of dorsal root ganglia (DRG) prepared from 7-9 days old chick embryos and/or explants of paravertebral sympathetic ganglia from 10-12 days old chick embryos, by evaluating the survival and differentiation of said explants in the presence of concentrations of rhNGF, or 2.5S mNGF, comprised between 1 and 100 ng/ml and after an incubation time comprised between 24-48 hrs. Differentiation and survival can be maintained in the presence of said concentrations of rhNGF up to about 2-3 weeks.

In an embodiment of the invention, the assay at point c. can be carried out by evaluating the phosphorylation of the high affinity trkA receptor by immunoprecipitation experiments. The peak of receptor activation can be observed after short times between 1 and 10 min., in particular after 5 min., and with concentrations of rhNGF, or 2.5S MNGF, comprised between 1 and 100 ng/ml, for example concentrations of 5-10 ng/ml can be used.

The biological activity of the rhNGF of the invention may be analyzed both in the conditioned culture medium of the rhNGF-producing cells properly diluted in the culture medium of PC12 cells or in ganglia to give the desired final concentrations. It can also be analyzed in the medium of the middle/large scale production systems in order to verify that the biological activity of the rhNGF produced in said systems is comparable, according to the indications given above, to those of the 2.5S mNGF. Production systems suitable for middle or large scale can be, for example, commercially available production systems like MiniPERM® (Greiner Bio-One, Germany), Roller Bottles or any other bioreactor system known to the technician of the field for middle or large scale production of recombinant proteins in mammalian cells growing in adhesion.

For the assay at point d. regarding the in vivo biological activity, newborn mice can be used. After injection of equal doses of beta rhNGF or 2.5S MNGF, usually 5 μg/gr of body weight for 5 consecutive days in parallel experiments, the biological activity of the recombinant neurotrophin produced can be then evaluated on: superior cervical ganglia (hypertrophy) and cutaneous tissues at the injection site (mast cells activation and regulation of Substance P and trkA levels).

The hypertrophy of SCG ganglia can be evaluated by comparing the effect of the beta rhNGF used to that of the murine neurotrophin by means of histological analysis of sections following standard fixing and staining processes, well known to the skilled person, such as for example toluidine blue staining.

Furthermore, it is possible to evaluate in the mice, also the presence/absence of symptoms of physical discomfort, usually present after the treatments as described above with the 2.5S mNGF or other rhNGF on the market and, instead, absent after the treatments with the beta rhNGF of the invention. This discomfort usually occurs as hyperalgesia localized at the injection site and hypersensitivity to thermal and mechanical stimuli. These are among the side effects commonly reported both in the in vivo studies and in patients enrolled in the clinical trials of peripheral neuropathies. Unlike the 2.5S NGF and the other mentioned rhNGF, the rhNGF of the invention does not induce these side effects that are shown in neonatal mice as hypersensitivity to thermal and mechanical stimuli as well in form of general uneasiness of the animal to the standard manipulation procedures.

The rhNGF of the invention, given its aforementioned characteristic biological activities and its human origin, is particularly suitable to be used as a medicament unlike the known commercial beta rhNGFs, that are clearly not suitable for medical use, as the trials carried out with said molecules have demonstrated that said molecules induced hyperalgesia and did not possess in vivo an effectiveness comparable to that of 2.5S murine NGF, regardless of the good results of the in vitro studies. For this reason, the beta rhNGF of the present invention is particularly advantageous. As clearly shown in tables 2 and 3, it shows in vivo activities that are comparable to the 2.5S murine NGF and, furthermore, in the tests performed on animal models in vivo it did not lead to symptoms of discomfort and pain in said animals unlike the known and/or commercially available rhNGF molecules used for the comparison studies. Tables 2 and 3 show also the same tests performed with the beta rhNGFs currently commercially available beta rhNGFs.

TABLE 2
BIOLOGICAL ACTIVITY OF THE rhNGF MOLECULE OF THE
INVENTION: COMPARISON WITH 2.5S mNGF AND OTHER TWO
COMMERCIALLY AVAILABLE NGF MOLECULES

| | | body | body | | | |
|---|---|---|---|---|---|---|
| | | weight | weigh at | | | |
| | | at birth | 9 days | Incisive | | In vitro |
| Molecule | Neurons/SCG | (gr) | (gr) | eruption | eyelid opening | effects |
| Control NI | 25.300 ± 350 | 5.3 | 27.4 | 11 | 14 | − |
| Control | 25.000 ± 367 | 5.4 | 27.5 | 11 | 14 | − |
| 2.5S | 40.260 ± 500 | 5.1 | 24.4 | 12 | 14 | +++ |
| mNGF | | | | | | |
| rhNGF | 30.570 ± 480 | 5.3 | 22.3 | 12 | 14 | +++ |
| Sigma | | | | | | |
| rhNGF | 28.640 ± 650 | 5.3 | 22.0 | 11 | 14 | ++(+−) |
| Alomone | | | | | | |
| rhNGF | 36.840 ± 575 | 5.4 | 26.5 | 11 | 14 | +++ |
| invention | | | | | | |

In vivo tests carried out on neonatal mice (n=5 each experimental group). Daily administration for 5 consecutive days with 5 μg/g body weight in 50 μl of saline solution. Some animals were sacrificed the day after the last injection to count the number of cells and evaluate neuronal survival; the other animals were sacrificed at post-natal day 9 to evaluate the effect of the molecules on the weight development, incisive eruption and eyelid opening.
In vitro tests carried out on sensory ganglia from chicken embryos; +++ indicates neurite outgrowth after 24 hr in culture equal to the length in diameter of the ganglion; ++(±) indicates neurite outgrowth after 24 hr in culture equal to 75% or less of the diameter of the ganglion.

The beta rhNGF of the invention also shows, as reported in Table 3, activities comparable to those of the 2.5S MNGF in several in vitro and in vivo tests.

TABLE 3

| | 2.5 mNGF | rhNGF | rhNGF | rhNGF |
|---|---|---|---|---|
| Test | (Promega) | invention | Alamone Labs | Sigma |
| PC12 and DRG | 100% | 100% | 100% | 100% |
| differentiation | | | | |
| TrkA | 100% | 100% | 100% | 100% |
| phosphorylation | | | | |
| SCG hypertrophy | 100% | 91.5% | 71% | 76% |

The process of the invention is particularly advantageous as it allows for the purification of the recombinant protein directly from the culture medium of the cells, without requirement for protein extraction from the transformed cells.

In fact, the fact that the beta recombinant hNGF produced is directly secreted into the culture medium is particularly advantageous as, in this way, processes for extraction of the recombinant protein from the cells are not required, thus avoiding the possibility of contamination of the product with undesired cellular material, such as for example the proNGF precursor. Indeed, the method of production of the invention is particularly efficient in processing the precursor pro-NGF and only mature rhNGF is secreted. In fact, the culture medium contains only the mature beta rhNGF and no unprocessed form of NGF is present therein. This property is of great relevance, especially in perspective of a therapeutic use of the beta rhNGF object of this invention as, according to recent data known in the art, the proNGF form preferentially interacts with the p75 NGF receptor thus triggering biological activities (apoptosis) that contrast the neurotrophic activity of NGF.

Therefore, the rhNGF of the invention does not induce apoptotic responses. Surprisingly, the beta rhNGF produced with the process of the invention, besides being produced in amounts higher or equal to 1 mg/L, also shows characteristic in vitro and in vivo activities, as indicated above, never reported up to date for molecules of beta rhNGF, and described, until today, only for the native 2.5S murine NGF. The method of the invention is, hence, particularly advantageous as it allows the abundant production of a recombinant human beta NGF having in vitro and in vivo biological activities comparable to those of the 2.5S murine NGF which has been, until today, the only molecule usable for medical purposes in exceptional situations, but that, given its murine origin, is not suitable for conventional pharmaceutical use.

For the carrying out of the invention, the cDNA of interest may be cloned by means of standard PCR techniques using, for example, primers that can be obtained with standard programs, capable to amplify the region of interest (exon 3), using the published human NGF sequence to design the primers. Then, the cDNA of interest (exon 3 of the human NGF gene, published in the literature, Ullrich et al., Nature 303: 821-825, 1983) can be inserted into vectors that allow the verification of the correct sequence of the insert which shall comprise the sequence coding for the 120 aa of the mature human NGF, the sequence coding for the 103 aa of the prosequence in the human proNGF, and the sequence coding for the 18 aa of the signal sequence of the native human NGF. Said cDNA can be subsequently sub-cloned into an appropriate vector.

In the method of the invention, said vector can be any vector known in the art and/or commercially available capable to express the inserted protein in mammalian cells. Among these: the pTRE vector (TetOff System, Clontech) or any other vector comprising a strong inducile promoter, such as for example the vectors of the pT-REx series (Invitrogen). The choice of a vector comprising a strong promoter, such as for example the CMV promoter, offers the advantage of guaranteeing high production of the protein of interest in eukaryotic cells. In particular, a tetracycline-dependent vector, guarantees maximum expression levels, much higher than those that can be obtained with a vector containing a constitutive CMV promoter. For instance, the PTRE vector (TetOff System, Clontech) contains, upstream of the minimal CMV promoter, seven repeats of a tetO sequence for binding of the regulatory protein tTA uncoded by the regulatory pTet-Off plasmid (Gossen M & Bujard H, Proc. Natl. Acad. Sci. USA 89: 5547-5551, 1992). This regulatory system ensures expression levels of the recombinant protein even higher than other inducible expression systems containing, besides the promoter, enhancer regions that are responsive to heavy metals or steroid hormones.

Other possible vectors suitable for expression in mammalian cells include the RheoSwitch system (NewEngland BioLabs), macrolide-inducible vectors, such as pTRIDENT, pDuoRex, pMF189, pMF229 (Weber W et al., Biotechnol. Bioeng. 80:691, 2002), ecdysone-inducible vectors such as the pEGSH (Stratagene).

Advantageously, the vector of the invention will also include at least a marker gene for an easy and successful selection of the transfected cells and, possibly, also a regulatory element.

In one embodiment of the invention, the amplified construct described above can be subcloned in a PTRE vector, (TetOff system, Clontech), downstream of the pCMV promoter present in the commercial vector, thus generating the pTRE-hNGF construct.

The mammalian cells of the invention can belong to any mammalian cell line, known to the skilled person, suitable for production of human proteins. Among these, as illustrative rather than limiting examples, are the HeLa cells, MEF, CHO, COS, BHK, HEK293, VERO cells, W138 and MDCK cell lines, or L929 fibroblasts, 3T3 fibroblasts, or other stabilized mammalian cell lines. Anyhow, whichever is the cellular system used, the cells shall be genetically modified to constitutively express, besides the plasmid vector comprising the human NGF cDNA, also the regulatory protein required by the inducible system of choice. Transformation of the mammalian cells with the appropriate expression vector as indicated above can be carried out by using any of the transfection methods known to the technician of the field, such as for example, electroporation, transfection by calcium-phosphate precipitation or liposomial complexes.

The selection of suitable cells according to the invention, can be obtained by verifying the abundant presence of beta rhNGF in the culture medium, and by analyzing said beta rhNGF by using the assays indicated above. By using this approach, the cellular clones obtained can be selected depending on the properties of the beta rhNGF produced and their capability to secrete said beta rhNGF.

Advantageously, cells secreting higher amounts of equally active recombinant protein will be chosen for the process of the invention, thereby making it possible to obtain, besides the advantage of the quality, also the advantage of the quantity of the product.

The beta rhNGF of the invention can be recovered, according to the production process of the invention, directly from the cell culture medium without the need of extraction from cells and thus greatly limiting the likelihood of contamination of the protein with cellular materials, such as, for example, unprocessed forms of NGF. The protein so obtained can be eventually purified by means of standard techniques known to the expert of the field.

In one embodiment of the invention, the purification of the protein can be obtained by using a modification of the method published by Allen (Allen et al. J. Biochem. Biophys. Methods 47: 239-255, 2001) by dialysis of the culture medium against 25 mM MOPSO pH 7.0 and a first passage on a ion-exchange chromatographic column (HiTrap SPFF, Amersham) coupled to a FPLC system (Pharmacia) followed by chromatography on hydrophobic matrix (Phenyl Sepharose, Amersham) or any other chromatographic system and/or gel filtration that can be appropriate for separation of the recombinant NGF from the serum proteins in the culture medium.

In a further embodiment of the invention, the cells selected at point III) of the process, can be cultured at point iv) in systems suitable for middle or large scale production of the beta rhNGF of the invention.

The embodiment herein indicated can be applied to all cells that can be selected according to the aforementioned process, obviously being particularly advantageous the use of the cellular clones selected at the point iii) of the process, that present the highest levels of production. They can be maintained as continuous cultures in 175 cm² flasks. Culture medium can be harvested at regular intervals of 1-3 days and used for both the quantitative analysis by NGF-ELISA or Western Blot, and the analysis of the biological activity, and for the purification process.

Another culture system that can be used for continuous production of rhNGF is given by minibioreactors (MiniPERM system, Greiner Bio-One, Germany) for mammalian cells growing in adhesion that allow to obtain high density cultures of mammalian cells. These systems are particularly useful for the scale up process (lab scale production).

The culture medium can be harvested from the production module at regular intervals of 1-2 days and used for both the quantitative analysis by NGF-ELISA or Western Blot, and the analysis of the biological activity, and for the purification process.

The purification process of the rhNGF can be carried out by any method known to skilled person, for example, starting from the medium conditioned by the highly productive clones maintained both in 175 cm² flasks and in minibioreactors. The purification process has been developed according to a modification of the method published by Allen (Allen et al. J. Biochem. Biophys. Methods 47: 239-255, 2001) by dialysis of the pool of medium against 25 mM MOPSO pH 7.0 and a passage on a ion-exchange chromatographic column (HiTrap SPFF, Amersham) coupled to a FPLC system (Pharmacia) followed by chromatography on hydrophobic matrix (Phenyl Sepharose, Amersham).

The in vitro and in vivo analyses of the biological activity of the beta rhNGF of the invention can be carried out by comparing the activities thereof to those of the 2.5S murine NGF in parallel assays, as indicated above, by means of standard techniques known to the person skilled in the art.

The beta rhNGF of the invention, given its particular properties, will be particularly suitable to be used as a medicament in general. In particular, in some embodiment of the invention, said molecule can be advantageously used for all those therapeutic applications for which the use of the neurotrophin NGF is believed to be suitable, such as for example Alzheimer's and peripheral neuropathies with different etiology, such as genetic predisposition, nutritional or dysmetabolic factors (like diabetes and alcoholism), viral infections (such as those caused by HIV), trauma and cytotoxic agents such as for example cytostatic drugs used for antitumoral therapy (cisplatin, taxol). Other therapeutic applications of the rhNGF include also cutaneous and corneal ulcers, vasculite or other diseases with high inflammatory or immune components such as multiple sclerosis.

Advantageously, hence, medicaments can be prepared comprising the beta rhNGF of the invention as the main active principle.

Therefore, according to the invention, it will be possible to prepare pharmaceutical compositions comprising the beta rhNGF of the invention, other possible active compounds when advisable, and pharmaceutically acceptable excipients which will vary depending upon the type of composition to be prepared. A very large number of forms of pharmaceutical compositions is already known to the skilled person, which may be liquid, solid, semi-solid, gel, powder form, lyophilized, suspension, etc. It will be clear to the person skilled in the art which excipients are the most suitable for the desired embodiment. For example, the compositions of the invention can be in injectable form, and in forms that allow the preferential transport toward specific organs or cell types by using carriers or by any other means known to the skilled person. In one embodiment, the beta rhNGF of the invention can be preferentially carried toward the central nervous system. For the central nervous system, for instance, an administration by intraventricular injection or nasal spray might be used (De Rosa R et al., Proc. Natl. Acad. Sci. USA 102: 3811-3816, 2005).

For peripheral neuropathies it might be administered by subcutaneous injections. For cutaneous and corneal ulcers and vasculite it might be applied in form of formulations for topical use.

The realization of the aforementioned compositions is known to the skilled person, and this kind of compositions is taught for example, in the Remington's Pharmaceutical Sciences, Mack Publishing Co., Easton, Pa.

In the methods of the invention, the beta rhNGF of the invention can be administered with suitable pharmaceutical diluents, excipients or vehicles (hereinafter referred as “excipients” in general) selected as being the most suitable to the desired form of administration according to the general knowledge of the pharmacopoeia.

The active principle of the invention can be also administered and prepared in form of liposomal distribution systems, such as small unilamellar vesicles, large unilamellar vesicles, and multilamellar vesicles. Said liposomes can be formed by a variety of phospholipids and steroids, such as cholesterol, stearylamine or phosphatidil-choline, for example.

The active principle of the invention might be distributed by using monoclonal antibodies as vehicles to which the molecule of the invention will be coupled. The beta rhNGF of the invention can be also coupled to soluble polymers in form of directional carriers. Examples of polymers include polyvinylpirrolidone, the copolymer pyrano, polyhidroxy-propyl-metacrylamide-phenol, the polyhidroetyl-aspartamide-phenol or others. Moreover, the active principle can be coupled to a class of biodegradable polymers useful for the controlled release of the drug, such as, for instance, polylactic acid, polyglycolic acid, copolymers of said acids, polyε-caprolactone, polyorthoesters, polyhydropirano and similar.

According to the present invention the aforementioned compositions will contain therapeutically effective doses of beta rhNGF. Clinical trials can be performed for each pathology in order to establish the best dose for each pathology starting from the doses already known from the literature, i.e. comprised between 0.01 and 100 μg/Kg body weight, for example between 0.1 and 0.3 μg/Kg body weight. The present invention also includes a therapeutic method for pathologies whose therapy requires the use of neurotrophins like NGF, such as, for example, Alzheimer's and peripheral neuropathies with different etiology, like genetic predisposition, nutritional factors or dysmetabolisms (like diabetes and alcoholism), viral infections (such as those caused by HIV), trauma and cytotoxic agents like, for example, cytostatic drugs used for the therapy of tumors (cisplatin, taxol). Other therapeutic applications of the rhNGF also include cutaneous and corneal ulcers, vasculite or other diseases with high inflammatory or immune component like multiple sclerosis, and include the administration of the rhNGF of the invention at therapeutically effective doses to patients in need thereof. According to said method the beta rhNGF of the invention can be administered in any of the formulations indicated above.

### EXAMPLES:

### Example 1

### Construction of the Expression Vector pTRE-hNGF

The cDNA of human NGF (exon 3) was cloned by the Polymerase Chain Reaction (PCR) method by using as template a human hippocampal cDNA library (Human Brain, hippocampus Marathon-Ready cDNA, Clontech) and a set of primers exactly complementary to the flanking regions of the cDNA fragment to be amplified and designed by using the sequence published by Ullrich (Ullrich A et al., Nature 303: 821-825, 1983).

The PCR reaction comprising the template DNA and primers was performed by using 28 cycles of 94° C. for 1 min, 55° C. for 2 min and 72° C. for 2 min in a DNA Thermal Cycler (Perkin-Elmer). The amplified cDNA fragment (about 800 bp) was first directly inserted into the vector pCR2.1-TOPO-TA (Invitrogen) by the 3′adenylated ends, and therefrom subcloned into HindIII/XhoI of the polylinker region of the pcDNA3.1 vector (Invitrogen).

For regulated production of rhNGF in mammalian cells, the human NGF cDNA was then subcloned into the plasmid vector pTRE (TetOff system, Clontech) by XbaI/SpeI digestion of the pcDNA-hNGF construct and ligation of the cDNA fragment into the unique XbaI site of the MCS immediately downstream of a pCMV promoter comprising a Tetracycline-regulated element (TRE) to generate the pTRE-hNGF construct.

All the constructs prepared during the process described in this invention were analyzed by sequencing and restriction analysis to determine the correct sequence and orientation of the NGF cDNA. The function of the pTRE-hNGF construct was evaluated by means of transient transfection experiments followed by quantitative and qualitative analyses of the recombinant protein produced.

### Example 2

### Transfection and Selection of Clones of HeLa TetOff and MEF TetOff Cells

HeLa TetOff and MEF TetOff cells (5×10⁵ cells) were seeded in dishes of 100 mm diameter in DMEM comprising 10% FBS and maintained overnight at 37° C., 5% CO². Culture medium was changed 4 hours before the transfection that was carried out by the calcium phosphate precipitation method by adding to the cells the transfection mixture (10 μg DNA in 1 M CaCl2, 140 mM NaCl, 5 mM KCl, 1 mM Na₂HPO₄, 6 mM dextrose, 25 mM Hepes). Cells were incubated overnight with the transfection mixture comprising the plasmid vector PTRE-hNGF described in the example 1 and the plasmid vector pTK-Hyg (Clontech) (pTRE-hNGF:pTK-Hyg ratio 10:1). After overnight incubation, the medium was then replaced with complete medium comprising the selection antibiotics: G418 (200 μg/ml) for the selective maintenance of the regulator gene pTetoff, hygromycin (400 μg/ml) for the selection of the double stable transfectants. After about 14 days, single clones of double stable transfectants were then isolated by using Cloning Rings and singularly amplified in 25 cm² flasks comprising complete medium.

### Example 3

### Analysis of Production Levels of Beta rhNGF of the Invention in the Selected Clones

To determine the levels of daily production, cells of each cellular clone (10⁵ cells/60 mm dishes) were cultured in complete medium. Conditioned medium was harvested after 24-48-72-96 hours, centrifuged to remove cells and debris in suspension and used for quantitative analysis of the beta rhNGF herein secreted. Cells in each plate were washed with cold PBS and lysed in lysis buffer (20 mM Tris pH 8.0; 137 mM NaCl; 1% Nonidet-P40; 10% glycerol; 1 mM DTT; 2 mM PMSF; 0.1 μg/ml leupeptin; 5 μg/ml aprotinin). After a 20 min incubation at 4° C., cellular debris were pelleted by centrifugation at 14,000 g for 10 min at 4° C. and protein concentration determined by the Bio-Rad Protein Assay (Bio-Rad). In parallel dishes, cells were instead harvested by trypsinization and counted with the Coulter Counter. Levels of production/secretion of beta rhNGF were then analyzed both by NGF-ELISA (Promega) and by Western Blot as described. Briefly, lysates (5 μg of total proteins) and culture media (5 μl in 1× loading buffer) were electrophoresed by SDS-PAGE (12%) followed by blotting onto nitrocellulose membrane and probing overnight at 4° C. with an antibody against the N-terminal region of mature human NGF (H-20, Santa Cruz Biotechnology). Detection of the NGF bands was obtained by using the ECL chemiluminescence system (Amersham). Quantization was obtained by densitometric analysis of the NGF bands and interpolation of the sample values on a standard curve obtained with known amounts of the 2.5S MNGF loaded on the same gel.

### Example 4

### Selection and Maintenance of the Highly Productive hNGF-HeLaTetOff and hNGF-MEFTetOff Clones

Stable clones of hNGF-HeLaTetOff and hNGF-MEFTetOff obtained in the example 2 and analyzed in the example 3 were maintained in culture in 25 cm² flasks in complete medium comprising the selection antibiotics. Conditioned medium was then analyzed as described in the examples 3, 6 and 7.

Among the several selected clones, following quantitative and qualitative analyses of the beta rhNGF produced, deposited clones included: hNGF-HeLa-BALM1 (Deposit N^{o} CBA PD 05004) which produces about 433+36 ng/ml (3-days cultures) corresponding to a daily production yields of 104.2+11.8 ng/10⁵cells per day; and the clones hNGF-MEF-BALM2 (Deposit N^{o} CBA PD 05002) and hNGF-MEF-BALM3 (Deposit N^{o} CBA 05003) which both produce about 2 μg/ml of beta rhNGF (3-days cultures) corresponding to a daily production yield of 191+7 and 278+21 ng/10⁵ cells per day for the hNGF-MEF-BALM2 and hNGF-MEF-BALM3, respectively. The results regarding the production and obtained by Western Blot (used as the main quantization method) are however comparable, if not some times underestimated versus those obtained by using a NGF-ELISA system.

These clones present, also, a higher duplication time compared to both their WT and Mock counterpart and other clones isolated during the process. The duplication time of the deposited clones hNGF-HeLa-BALM1, hNGF-MEF-BALM2 and hNGF-MEF-BALM2 is in the range of about 32 hr, whereas the duplication time of the HeLa TetOff WT and MEF TetOff WT is about 21-22 hr.

### Example 5

### Lab Scale Production of Beta rhNGF in Minibioreactors

For continuous production of beta rhNGF in minibioreactors (MiniPERM system, Greiner Bio-One, Germany), cells of the clone hNGF-HeLa-BALM1 (8-10×10⁶ cells) were seeded on the membranes of the production module of the MiniPERM in DMEM supplemented with 5% FBS and the culture medium (35 ml) harvested every 24-48 hr and replaced with fresh medium. The medium in the nutrient module was instead changed every 4-6 days. Minibioreactors were kept under rotation on a Tuning device placed in the incubator a 37° C., 5% CO₂. The pool of media harvested at regular intervals was then used for both quantitative analyses and biological activity assays as described in the examples 3, 6 and 7, as well for the purification process.

Culturing the cells hNGF-HeLa-BALM1 in this system allowed to obtain a daily production of beta rhNGF ranging between 224 and 1550 μg (micrograms) and thereof obtain (in the several MiniPERM used) a total amount of recombinant protein ranging from 7.8 to 10.36 mg in about 2 weeks, about 100 fold higher than that obtainable by using conventional culture systems (flask), and with an average concentration of 20.3+3 mg/L.

### Example 6

### Differentiation of PC12 Cells and Dorsal Root Ganglia (DRG)

PC12 cells (7×10⁴) were seeded in dishes of 35 mm diameter in DMEM comprising 0.5% FBS/1% HS and appropriate dilutions of the medium conditioned by the beta rhNGF-producing clones were done to give final concentrations comprised between 1 and 100 ng/ml. The morphological modifications induced by the beta rhNGF object of this invention were then observed under an reversed microscope equipped with an Olympus camera.

For the analysis of the neurotrophic activity of the beta rhNGF on DRG explants, ganglia were dissected from 8-9 days old chicken embryos and placed into Hepes buffer (HBSS) and then cultured in 35 mm dishes coated with poly-L-lysine (1%) in DMEM comprising 10% FBS, 2 mM L-glutamine, 100 μg/ml streptomycin, 100 U/ml penicillin. Treatment of ganglia with the rhNGF produced in this invention or with the 2.5S mNGF (used as positive control) was performed soon after the preparation of the ganglia and repeated every 48 hr for about 14-21 days. The effect of the beta rhNGF on survival and differentiation of ganglia was observed with concentration of rhNGF as low as 0.1 ng/ml, it was evident after 24-48 hr incubation and could be prolonged for about 2-3 weeks.

### Example 7

### Phosphorylation of the trkA Receptor

The biological activity of the beta rhNGF object of this invention was evaluated by determining also the capability to activate the phosphorylation of the high affinity trkA receptor. To this end, immunoprecipitation experiments were performed as described in Colangelo (Colangelo et al., Glia 12: 117-127, 1994). Briefly, PC12 cells (10⁶/100 mm dishes) were incubated for 5 min in DMEM supplemented with 0.5% FBS/1% HS and containing rhNGF or 2.5S mNGF at concentrations comprised between 1 and 20 ng/ml, washed with cold PBS and lysed at 4° C. in 1 ml of RIPA buffer (50 mM Tris pH 7.5, 150 mM NaCl, 1% Nonidet P-40, 0.5% sodium deoxycholate, 0.1% SDS and 1 mM DTT) containing proteases (2 mM PMSF, 1 μg/ml leupeptin, 5 μg/ml aprotinin) and phosphatases (10 mM NaF e 1 mM di sodium orthovanadate) inhibitors. Lysates (300 μg total proteins) were incubated overnight at 4° C. with 2 μg of rabbit anti pan-trk IgG (C-14, Santa Cruz Biotechnology) and precipitated with protein A-Sepharose (Sigma) for 2 hr at 4° C. Immunocomplexes were then washed with lysis buffer, resuspended in loading buffer (50 mM Tris pH 6.8, 2% sodium dodecyl sulphate, 100 mM DTT, 10% glycerol, 0.1% bromophenol blue), separated by 7.5% SDS-PAGE and transferred onto nitrocellulose membrane. Blots were then probed overnight at 4° C. with an anti-phosphotyrosin mAb (PY99, Santa Cruz Biotechnology) in TBST, followed by incubation with a HRP-conjugated donkey anti-mouse IgG (Jackson Immunoresearch; 1:10,000) for 1 hr at room temperature. The phospho-trkA bands were then detected by using the ECL system (Amersham). Quantization of the p-trkA species was obtained by densitometric scanning of the p-trkA bands by using the Scion Image software. The activity was evaluated in terms of percentage of induction versus control (untreated cells).

The results in FIG. 3 indicate that the rhNGF induces trkA phosphorylation levels comparable to those induced by equal concentrations of 2.5S mNGF.

### Example 8

### In Vivo Studies

The biological activity of the rhNGF of the invention was also evaluated by in vivo studies aimed at determining not only its neurotrophic activity but also evaluate the absence of interferences with the normal neonatal development and/or the absence of side effects currently reported with other rhNGFs. To analyze the in vivo activity, newborn mice (5 animals each group) were injected with the rhNGF of the invention or with 2.5S mNGF at doses of 5 μg/gr body weight. Treatments were performed daily for 5 consecutive days. As control, some animals were treated with an equal dose of cytochrome C (CY), a molecule possessing chemical-physical properties similar to NGF but devoid of biological activity, whereas another control group did not receive any treatment in order to rule out any effect due to the stress of the injection. Following treatments, some animals were sacrificed the day after the last administration (on the 6^{th} day) to evaluate the effect of rhNGF treatments on the development of cervical ganglia, activation of mast cells and regulation of Substance P and trkA levels in cutaneous tissues at the injection site. To this end, treated and untreated animals were sacrificed and superior cervical ganglia (SCG) and cutaneous tissues immediately dissected out, fixed in 4% paraformaldheyde in phosphate buffer, stained with toluidine blue and mounted in toto for macroscopic analysis. Then, ganglia and cutaneous tissues were sectioned and stained with toluidine blue. For each ganglia the count of total neurons was performed, as well neuronal morphology and survival were analyzed. In the cutaneous tissues, instead, the effect of the NGF molecules on trkA and Substance P (SP) expression levels were examined, as well the distribution and degranulation of mast cells.

Some animals were instead sacrificed at post-natal day 9 to evaluate the effect of the molecules on the weight development, incisives eruption and eyelid opening, in order to rule out a negative effect of the rhNGF of the invention on the normal post-natal development.

### Example 9

### Comparison of the rhNGF of the Invention with Other Commercially Available rhNGF (Alomone Lab. and Sigma)

The results in table 3 were obtained by evaluating, in the different tests indicated (PC12 cells and DRG differentiation, trkA phosphorylation and hypertrophy of ganglia), the activity of the rhNGF of the invention and, as a comparison, that of other two commercial rhNGF (Alomone Laboratories and Sigma) at different concentrations comprised between 0.1 and 20 ng/ml, and precisely at the concentrations of 0.1, 1, 2.5, 5, 10, 20 ng/ml. Activities were calculated as percentage of that induced by equal concentrations of 2.5S murine NGF.

Data reported in table 3 indicate that in vitro the rhNGF of the invention presents an activity comparable to that of the other commercial rhNGFs, whereas a marked difference of activity was observed essentially in the in vivo studies. In fact, the rhNGF of the invention presents a neurotrophic activity of about 92% of the 2.5S mNGF, compared to 71% of the rhNGF Alomone Lab. and 76% of the rhNGF Sigma. In addition, unlike the other two rhNGF, the rhNGF of the invention does not induce typical side effects, such as partial body weight reduction and hyperalgesia.

However, some differences were indeed found also in the in vitro studies.

The first difference is that, although the three rhNGF, at equal doses, induce in PC12 cells the same levels of trkA phosphorylation and differentiation in terms of number of differentiated cells and length of neurite processes, the neurotrophic activity of the rhNGF of the invention is characterized also by hypertrophy of the differentiated cells (larger cell body) whereas, at equal doses, the other rhNGF induce also the apoptosis of a certain number of cells. This effect, if on one side is typical of a certain toxicity at high doses of NGF (50-100 ng/ml), on the other hand it might be due to the presence of unprocessed forms of NGF like the proNGF. In fact, according to recent data in the literature, the proNGF interacts preferentially with the p75 receptor that, in virtue of the presence of a death domain in the cytoplasmic region, is able to activate an apoptotic cell death process.

To this end, the different rhNGF, at the same dilutions used for the treatments, were analyzed by Western Blot, both to verify that the amount used for the treatments were similar for the three rhNGF, as the preparations were from different laboratories and quantization could not perfectly match, and also to verify the presence or less of unprocessed forms of proNGF.

Indeed, Western Blot analysis indicated that equal amounts of the rhNGF Alomone Lab. and Sigma, calculated according to the indications on the vials, showed a different immunological activity, compared to the rhNGF of the invention, toward the anti-NGF antibody H-20 (Santa Cruz Biotechnology) which recognizes the N-terminus of mature human NGF, said antibody being used also for Western Blot analysis of the rhNGF of the invention as described in the example 3.

Moreover, the rhNGF of Alomone Lab. and Sigma presented additional bands of both lower molecular weight that might be the product of partial degradation and, the rhNGF Sigma, also bands of higher molecular weight, some of about 25 kDa that are more evident in the Western Blot performed with higher amounts of protein, and that might be likely due to homodimers of the molecule.

1-17. (canceled)

18. Beta subunit of recombinant human Nerve Growth Factor (rhNGF) being the expression product of the cDNA sequence of about 800 bp encoding the exon 3 of the human NGF gene, and showing biological activities higher than 76% of the biological activities of the 2.5S subunit of the native murine NGF in the:
a. test of evaluation of PC12 pheochromocytoma cells differentiation;
b. test of evaluation of survival and differentiation of dorsal root ganglia (DRG) and/or sympathetic paravertebral ganglia from chick embryos;
c. test of evaluation of phosphorylation of trkA receptor and;
d. test of evaluation of the induction of superior cervical ganglia (SCG) hypertrophy.

19. Beta rhNGF according to claim 18, wherein said biological activities are comprised between 80 and 100% of those given by the 2.5 subunit of the native murine NGF in the tests according to claim 18.

20. Beta rhNGF according to claim 18, wherein said biological activities are comprised between 90 and 100% of those given by the 2.5 subunit of the native murine NOF in the tests according to claim 18.

21. Pharmaceutical compositions comprising the beta rhNGF according to claim 18 and pharmaceutically acceptable excipients.

22. Pharmaceutical compositions according to claim 21 in liquid, solid, lyophilized form, in powder, in suspension, as liposomes.

23. Pharmaceutical compositions according to claim 22 wherein said pharmaceutically acceptable excipients are suitable for injectable formulations.

24. A medical treatment for the therapy of pathologies requiring the administration of neurotrophins wherein the beta rhNGF according to claim 18 is administered in therapeutically effective doses to patients in need thereof.

25. The medical treatment of claim 24, wherein the administering of therapeutically effective doses of beta rhNGF does not provoke the side effects hyperalgesia or allodynia.

26. A process for the preparation of beta rhNGF as defined in the claim 18 comprising the following steps:
i) constructing an expression vector suitable for expression in mammalian cells and comprising a EDNA sequence encoding the exon 3 of the human NGF gene, said cDNA sequence including a sequence encoding the beta chain of the mature human NGF, a sequence encoding the prosequence of the beta chain of human NGF and a sequence encoding the signal sequence of the beta chain of the human NGF.
ii) transforming mammalian cells with said vector;
iii) selecting cellular clones that are capable to secrete beta rhNGF having biological activities comprised between 90 and 100% of those given by the 2.5S subunit of the native murine NGF in the:
a. test of evaluation of PC12 pheochromocytoma cells differentiation;
b. test of evaluation of survival and differentiation of dorsal root ganglia (DRG) and/or sympathetic paravertebral ganglia from chick embryos;
c. test of evaluation of phosphorylation of trkA receptor and;
d. test of evaluation of the induction of superior cervical ganglia (SCO) hypertrophy.
iv) culturing of the cells selected at point iii) and recovering said beta rhNGF directly from the cell culture medium.

27. A process according to claim 26, wherein point iv) is carried out in a system for cell culture on middle or large scale.

28. A process according to claim 27 wherein said system is a bioreactor.

29. A process according to claim 26, wherein the yield of rhNGF is higher or equal to 18±3 mg/L in about 15 days.

30. A process according to claim 26, wherein said expression is regulated by a strong promoter comprised in said vector.

31. A process according to claim 26, wherein said mammalian cells are HeLa, MEF, CHO, COS, BHK, HEK293 cells.

32. Cells obtainable by the process according to claim 26.

33. Cells according to claim 31, deposit number CBA PD 05004, CBA PD 95002, CBA PD 05003.
